(19) United States
(12) Patent Application Publication
(10) Pub. No.: US 2022/0412992 A1
(43) Pub. Date: Dec. 29, 2022

(54) METHOD FOR MAKING A FINDING FOR THE FUNCTIONALITY OF AN ANOREXIGENIC SIGNAL PATH FOR A PATIENT

(71) Applicant: Labor Berlin - Charité Vivantes Services GmbH, Berlin (DE)

(72) Inventors: Tina BUCHHOLZ, Berlin (DE); Nancy KRÜGER, Brandenburg (DE); Oliver BLANKENSTEIN, Berlin (DE); Peter KÜHNEN, Berlin (DE); Falko BÖHRINGER, Berlin (DE)

(73) Assignee: Labor Berlin - Charité Vivantes Services GmbH, Berlin (DE)

(21) Appl. No.: 17/780,983

(22) PCT Filed: Nov. 27, 2020
(86) PCT No.: PCT/EP2020/083630

§ 371 Date: May 29, 2022

(30) Foreign Application Priority Data
Nov. 29, 2019 (DE) 10 2019 218 597.5

(51) Int. Cl. G01N 33/68; G01N 33/74

(52) U.S. Cl. CPC G01N 33/6893; G01N 33/74; G01N 2800/044

(57) ABSTRACT

The present invention relates to a method of providing an FAS finding (30) for the functionality of an anorexigenic signal path for a patient (1). Said method comprises the following steps: placing the patient (1) in a normalised preparation state in preparation for a normalised sample collection, providing a normalised sample matrix (10) collected from a patient (1) who was in the normalised preparation state, and determining at least one FAS indicator (11, 12, 13) from the normalised sample matrix (10), generating the FAS finding (30) based on the at least one determined FAS indicator (11, 12, 13).

The present invention relates to a method of providing a finding for the functionality of an anorexigenic signal path for a patient or subject. On the basis of such a finding, conclusions can be drawn regarding a possible genetically influenced obesity of the patient.

According to current studies, it is assumed that one in three people worldwide is overweight or obese. In cases of excess weight which can be harmful to health, one speaks of obesity. Obesity is now regarded as a chronic disease which is associated with an impaired quality of life and a high risk of secondary diseases. Not only can those affected suffer from physical consequences, they are often victims of discrimination within the population.

The causes of obesity vary. In addition to excessive calorie intake resulting from unhealthy diets, obesity can also be caused or at least influenced by genetic factors. According to the International Classification of Diseases and Related Health Problems, obesity is classed under the endocrine, nutritional and metabolic diseases.

Body mass index (BMI) serves as a rough measure for determining obesity. The BMI is calculated by dividing the body weight in kilograms by the height in metres squared. If the BMI is over 30 kg/m², this is classed as obesity according to the World Health Organisation. The BMI also allows classification into different degrees of obesity. Thus, with a BMI of between 30 and 34.9 one speaks of grade I obesity, with a BMI of between 35 and 39.9 of grade II obesity and with a BMI of 40 and more of grade III obesity or obesity permagna or morbid obesity. However, BMI is only a rough guide value. A BMI-based diagnosis is not sufficient for a targeted treatment of affected patients. In particular, the BMI does not allow a distinction to be made between excess weight due to a disturbed energy balance and an obesity which may be caused by genetic factors.

In order to produce a clinically reliable finding regarding a possible genetically caused or influenced obesity of the patient, an attempt is therefore made to obtain meaningful indicators on the basis of body substances of the patient. In practice, however, this has proved difficult, in particular with regard to a desirable standardisation of the indicators obtained.

The object of the present invention is to take into account at least partially the problems described above. In particular, it is the object of the present invention to create an improved method for generating a meaningful and consistently reproducible finding with regard to a possibly genetically caused obesity.

The above object is achieved by the claims. In particular, the above object is achieved by the method according to claim 1. Further advantages of the invention emerge from the dependent claims, the description and the drawings.

According to a first aspect of the present invention, a method of providing a FAS finding for the functionality of an anorexigenic signal path for a patient is provided. The method comprises the following steps:
placing the patient in a normalised preparation state in preparation for a normalised sample collection,
providing a normalised sample matrix collected from a patient who was in the normalised preparation state, and
determining at least one FAS indicator from the normalised sample matrix,
generating the FAS finding based on the at least one determined FAS indicator.

FAS is to be understood here as an abbreviation for the functionality of an anorexigenic signal path. Accordingly, the FAS finding is to be understood as a finding regarding the functionality of an anorexigenic signal path. In other words, the FAS finding can be understood as an assessment of the function of an anorexigenic signal path. A FAS indicator is, accordingly, to be understood as an indicator for assessing or diagnosing the functionality of an anorexigenic signal path. The FAS finding can therefore be arrived at with the help of the FAS indicators. Depending on the functionality of the anorexigenic signal path, the patient can achieve the desired feeling of satiety at different times, which helps them maintain a regulated diet. If the anorexigenic signal path is disturbed, this can lead to the patient not experiencing any, or only a reduced feeling of satiety and thus being more at risk of obesity than a person whose anorexigenic signal path is undisturbed or whose anorexigenic signal path functions as desired.

In the context of the present invention, it was recognised that the finding regarding the functionality of the anorexigenic signal path reliably allows direct conclusions to be drawn regarding an obesity, in particular a genetic obesity. It was also recognised that in order to obtain a reliably usable FAS finding, it is of decisive importance that the patient is in a normalised preparation state during sample collection, i.e. according to the invention, only a sample matrix from a patient who was in a predefined preparation state during sample collection is used. These measures make it possible, as desired, to generate the meaningful and consistently reproducible FAS finding with regard to the possible genetic obesity.

The generation of a FAS finding can be understood to mean a finding under which in the present case medically relevant physical or psychological phenomena, circumstances, changes and/or conditions of the patient relating to the functionality of their anorexigenic signal path are collected. Providing the normalised sample matrix can be understood to mean providing at least one normalised sample matrix. Patient can also be understood here to refer to a test subject.

The method for generating the FAS finding is in particular carried out non-invasively. That is to say, the method is not carried out directly on the human body. In preparation for the normalised sample collection, a non-invasive finding can be arrived at with regard to an infection and/or an inflammatory condition of the patient, wherein, depending on the finding, the patient is cleared for normalised blood sample collection. Experiments conducted within the context of the present invention have shown that the sample matrix may be useless in the case of an infection and/or an inflammatory condition of the patient.

The preparatory measure described above can therefore prevent an unnecessary and/or unusable FAS finding. This can save the time required for this purpose and corresponding costs.

In the context of the method according to the invention, a plurality of different FAS indicators are preferably derived from the normalised sample matrix. In particular, at least one first FAS indicator and at least one second FAS indicator are derived from the sample matrix, wherein the at least one second FAS indicator is different from the at least one first FAS indicator. The FAS finding can then be generated using an indicator spectrum comprising the at least one first FAS indicator and the at least one second FAS indicator.

The joint consideration of a plurality of different FAS indicators allows a significant gain in information or a correspondingly high accuracy of the FAS finding to be achieved. The indicator spectrum can therefore be understood to mean a collection of different FAS indicators. Accordingly, according to the invention a plurality of different FAS indicators are considered jointly.

It is generally difficult to achieve a reliable diagnosis on the basis of a single indicator chemically in the laboratory, because often the technical detection limit of a detection method lies very close to a lower end of the normal range and as a rule there is little space available below this for assessing a clearly pathological situation. According to the invention, a better or more accurate result is now derived from the overall view of the indicator spectrum than would be possible on the basis of a sum of individual findings.

With knowledge of a predefined symptomatology and further diagnostic measures, for example involving imaging, clinical symptoms or genetics, patterns characteristic of individual diagnoses can be identified from a larger number of analyses on the basis of the indicator spectrum. These can then be applied to the patient, without detailed knowledge of the broader diagnostic measures, to predict the diagnosis, the further progression of the condition or, for example, also the response to certain drug treatments. These patterns contain laboratory results which are conspicuous, not only in the sense of classical laboratory analysis.

The at least one first FAS indicator differs, in particular in its nature, from the at least one second FAS indicator. That is to say, the at least one first FAS indicator differs not only in amount and/or scope from the at least one second FAS indicator. That the indicator spectrum comprises the at least one first FAS indicator and the at least one second FAS indicator is to be understood to the effect that the respective FAS indicator can basically comprise an unlimited number of different FAS indicators. Thus, the indicator spectrum can also comprise three or more FAS indicators which are in each case different from each other.

As part of the FAS finding, an “MSH deficiency” or an α-MSH deficiency and/or a “MSH excess” are determined, which can then provide indications of a response to a therapy with, for example, α-MSH analogues. Both cases indicate a disturbance of the anorexigenic signal path. An MSH deficiency can be treated using an already existing α-MSH agonist, for example a synthetic peptide hormone, which reactivates a signalling cascade that was interrupted by the α-MSH deficiency. With the help of the present method, not only can the existence of an MSH deficiency or an MSH surplus be determined, this can also be determined quantitatively.

According to a further development of the method according to the invention it is possible that the patient is subjected for a defined period of time to an exclusion of light, in particular an exclusion of sunlight. In experiments conducted in connection with the present invention, it has been found that the exclusion of sunlight is particularly helpful in achieving the desired standardisation. This approach makes it possible to develop correspondingly meaningful FAS indicators, which in turn has a positive effect on the desired FAS finding.

In a method according to the present invention, the exclusion of light is preferably carried out for at least 10 hours, in particular for at least 20 hours. Extensive experiments conducted in connection with the present invention have shown that a minimum duration of 10 hours, preferably a minimum duration of 20 hours, has a particularly advantageous effect on the at least one FAS indicator which to be determined.

In addition, in a method according to the invention it is possible that the patient is placed in the normalised preparation state in preparation for a normalised blood sample collection, wherein the normalised sample matrix is a plasma sample. It has proved possible to achieve particularly meaningful results using a plasma sample. Nevertheless, other sample matrices can also be used for the present method. Thus, a blood sample, for example in the form of a whole blood sample, a serum sample, a cerebrospinal fluid sample and/or a urine sample, in each case in liquid or dry form, can also be used. When using dried sample matrices, the analytes, as explained above, are extracted from the dried sample matrix using suitable extraction agents, wherein a rehydration can be performed with or without organic solvent content. When dried blood is used, an enzymatic cleavage of the analytes can be expanded into peptide fragments.

According to a further embodiment of the present invention, in preparation for the sample collection, the patient can be required to fast for a defined period of time. This, too, has proven, in experiments conducted in connection with the invention, to be surprisingly effective in placing the patient in a normalised preparation state in order to obtain the desired FAS indicator. In a method according to the invention, the fasting is preferably carried out for at least 6 hours, in particular within a time window of 12 hours to 36 hours. In experiments, this period has been shown to be sufficient to achieve the desired results for the FAS finding.

In a method of the present invention, it is possible that the at least one first FAS indicator is determined on the basis of at least one measured value for at least one analyte in the sample matrix. Taking into account the at least one analyte, a particularly meaningful FAS indicator can be provided. In particular, a plurality of FAS indicators are determined on the basis of a plurality of measured values for a plurality of different analytes in the sample matrix. It is possible that a plurality of FAS indicators are determined, wherein the at least one first FAS indicator is determined on the basis of at least one measured value for a first analyte in the sample matrix and/or the at least one second FAS indicator is determined on the basis of at least one measured value for a second analyte in the sample matrix, wherein the first analyte and the second analyte are in particular located at different points within a control loop or a synthesis chain. The advantage of this approach is that the functionality of the control loop or the corresponding signal cascade can be measured and proven at different points. The individual analytes are interdependent, that is to say if one analyte is high or low, the other must also be correspondingly high or low. In this way, the desired results can be secured. The analytes, which can be derived from both liquid and dried sample matrices, can be extracted from the sample matrix by immunoprecipitation (IP) or solid phase extraction (SPE). The analytes can be bound to the column material and isolated by in each case selecting a suitable antibody or according to physicochemical properties using a C18 material under reversed phase conditions. A clean extract can be obtained by washing the analytes with aqueous solvents with subsequent elution through organic solvents. The extracted analytes can subsequently be concentrated by evaporation of the organic solvent and reconstituted in an aqueous solution. The extracted analytes can be separated from other matrix components of the sample using reversed phase chromatography in an online LC-MS/MS method. The extracted analytes can also be chemically altered, in particular derivatised. Methods such as the Immunoaffinity Chromatography (IAC) and the Hydrophilic Interaction Liquid Chromatography (HILIC) are also possible. A mass spectrometric analysis can be performed in a multianalyte method in MRM mode (multiple reaction monitoring), in which specific mass transitions of double or triple-charged analyte ions can be fragmented in the collision cell of the mass spectrometer and the fragment ions can be detected. By doping stable isotope-labelled internal standards, the quantitative determination of the analyte can be carried out by means of external calibration.

It can be of further advantage if, in a method according to the invention, the analyte is extracted from the sample matrix chromatographically or by immunoprecipitation and determined by means of a subsequent mass spectrometry. The mass spectrometry and the immunoprecipitation have proven to be advantageous in the present method. The advantage in comparison with an immunoassay is, for example, that a plurality of analytes can be measured simultaneously, for example using a multiplex method. On the other hand, cross-reactivities, such as often occur in immunoassays for example, can be excluded.

In a method according to the present invention, it is moreover possible that the analyte is MSH, in particular α-MSH. Neuronal α-MSH is formed in the hypothalamus and anterior pituitary lobe from the proprotein proopiomelanocortin (POMC). Hypothalamic α-MSH plays a central role in weight regulation in that, on the basis of signals from the periphery, the fat content of the body and the stomach filling lead to the release of α-MSH, which conveys the feeling of satiety via the central melanocortin receptors. Mutations in the POMC gene can lead to a deficiency of the proprotein and thus of the α-MSH. Thus, due to the interrupted signal chain, no satiety signal can be generated. This can lead to uncontrolled, greatly increased food intake and thus to extreme obesity. In experiments conducted in connection with the present invention, it was shown that satiety is associated with an increase in the α-MSH concentration, in particular in the cerebrospinal fluid. Defects in this central α-MSH signal transmission have so far only been diagnosed on the basis of the underlying genetic defects, on the one hand because the sampling of cerebrospinal fluid is associated with a high effort and risk; on the other hand, in the case of a proven genetic defect the diagnosis is considered reliable and does not need to be further confirmed in cases of extreme obesity, given consistent clinical symptoms. Therefore, the detection of α-MSH did not previously play a role in the consideration of extreme obesity. While the failure of the central production of α-MSH as well as defects of the MC4 receptor are clearly associated with early manifest extreme obesity, hardly any data is available on possible clinical effects of the modulation of this appetite regulation mechanism. However, it has now been recognised, in experiments conducted in connection with the present invention, that in extremely overweight people who suffer from a disturbance in the signalling cascade between the formation of leptin in peripheral adipose tissue up to the melanocortin receptor, a reduced α-MSH effect in the hypothalamus contributes causally to obesity. A common feature of all these disturbances would be a reduced central α-MSH production or a defect in an MC4 receptor. Thus, a particularly meaningful FAS finding can be generated on the basis of the α-MSH concentration or on the basis of the determination of the analyte in the form of α-MSH.

Furthermore, in a further development according to the invention, the at least one FAS indicator can comprise a CLIP concentration in the normalised sample matrix. A CLIP concentration can be understood to mean the concentration of corticotropinlike intermediate peptide (CLIP). The amount of CLIP formed, the amount of which may be equimolar to the α-MSH concentration, allows an indirect measurement of the α-MSH concentration. Accordingly, one is not reliant on the direct measurement of the α-MSH concentration. By measuring the α-MSH concentration, the aforementioned advantages with regard to the desired diagnosis can in turn be achieved. Conclusions regarding the desired FAS finding can also be drawn directly on the basis of the CLIP concentration.

It is also possible that, in a method according to the invention, the at least one FAS indicator comprises the concentration of at least one peptide hormone in the normalised sample matrix. By determining the concentrations or the ratios of relevant peptide hormones or at least one peptide hormone, diagnostic conclusions can be drawn regarding the FAS finding. In a similar way as described above with regard to the CLIP concentration, the α-MSH concentration in the blood which is to be determined can be determined indirectly by measuring relevant peptide hormones. The concentration of the at least one peptide hormone is preferably determined by a multiplex method. It can be of further advantage if, in a method according to the invention, the at least one FAS indicator in each case comprises the concentration of different peptide hormones in the sample matrix. As a result, a high accuracy of the FAS finding can be achieved.

Further measures to improve the invention are disclosed in the following description of various exemplary embodiments of the invention, which are represented schematically in the figures. All features and/or advantages resulting from the claims, the description or the drawing, including constructive details and spatial arrangements, can be essential to the invention both in themselves and in the various combinations.

In each case schematically:

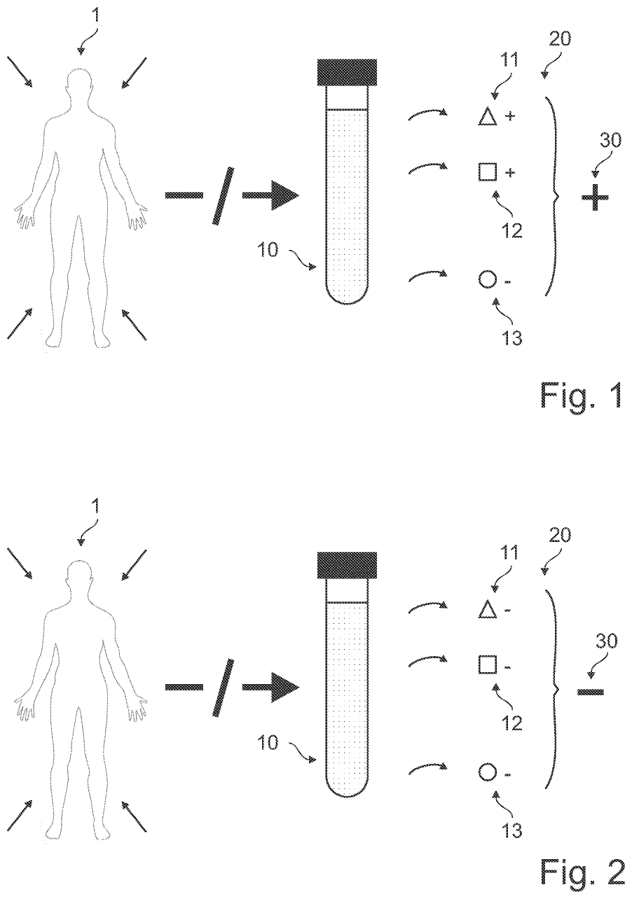

FIG. 1 shows a representation explaining a method according to a first embodiment of the present invention,

FIG. 2 shows a representation explaining a method according to a second embodiment of the present invention,

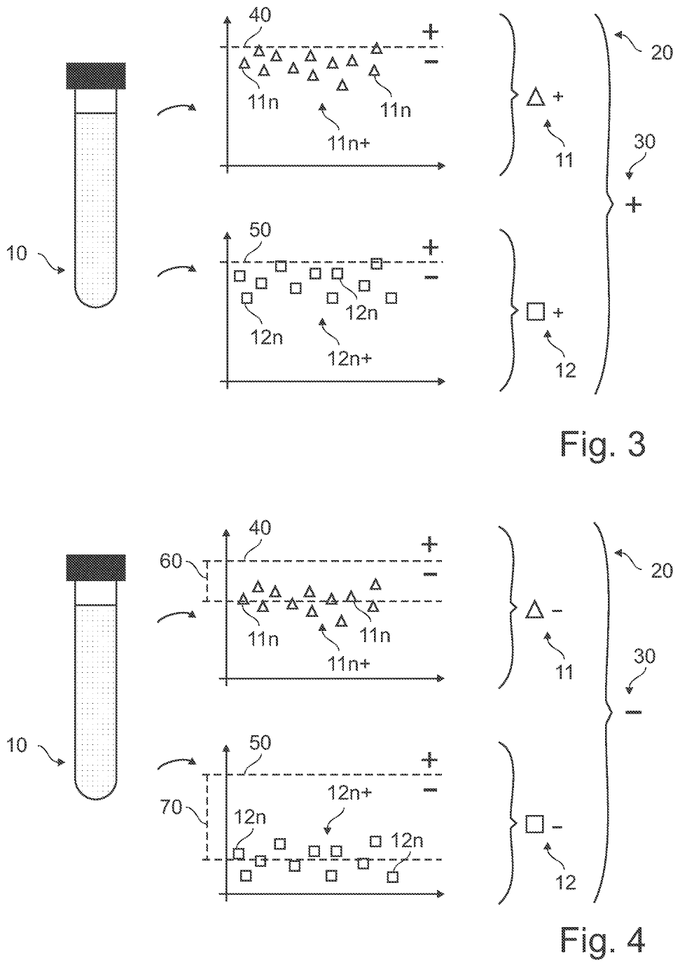

FIG. 3 shows a representation explaining a method according to a third embodiment of the present invention,

FIG. 4 shows a representation explaining a method according to a fourth embodiment of the present invention.

Elements with the same function and mode of action are in each case given the same reference signs in FIGS. 1 to 4.

A method of providing a FAS finding 30 for the functionality of an anorexigenic signal path for a human patient 1 according to a first embodiment is now explained with reference to FIG. 1. The patient 1 represented in FIG. 1 is first placed in a normalised preparation state in preparation for a normalised blood sample collection. In preparation for the sample collection, the patient is subjected to an exclusion of sunlight for approx. 24 hours. In preparation for the sample collection, the patient is also is required to fast during these 24 hours.

A normalised sample matrix 10 is then provided in the form of a blood sample which was collected from a patient 1 who was in the normalised preparation state. According to the example shown in FIG. 1, three different FAS indicators 11, 12, 13 are then derived from the normalised sample matrix 10. The three FAS indicators 11, 12, 13 form an indicator spectrum 20. Based on the predominantly positive indication, a FAS finding 30 can now be derived from the overall consideration of the indicator spectrum 20 to the effect that there is probably no, or only a very weakly manifested, malfunction of the anorexigenic signal value. In other words, the anorexigenic signal value appears to function normally or substantially normally. As a result, it can now in turn be concluded that an obesity in question is not, or only scarcely, caused by genetic factors. The illustrated marking of the FAS indicators 11, 12, 13 and of the FAS finding 30 with “+” and “−” also allows exactly the opposite diagnosis, depending on a previously specified interpretation of the sign.

FIG. 2 shows an example according to a second embodiment in which all FAS indicators 11, 12, 13 come up negative, i.e. according to a specified interpretation they indicate a malfunction of the anorexigenic signal value. By considering the associated indicator spectrum 20, a FAS finding 30 can now be derived which indicates a genetic malfunction of the anorexigenic signal value.

A method according to a third embodiment is now explained with reference to FIG. 3. The FAS indicators 11, 12 shown in FIG. 3 are in each case determined on the basis of a measured value 11n, 12n for an analyte in the sample matrix. More precisely, according to the embodiment shown, a plurality of first measured values 11n are determined for a first analyte in the sample matrix 10 and a plurality of second measured values 12n are determined for a second analyte in the sample matrix 10, wherein the first measured values 11n are expanded to form a first group of measured values 11n+ and the second measured values 12n are expanded to form a second group of measured values 12n+. The first FAS indicator 11 is determined on the basis of the first group of measured values 11n+ and the second FAS indicator 12 is determined on the basis of the second group of measured values 12n+. The first analyte and the second analyte are located at different points within a control loop or a synthesis chain.

In the present case, the first FAS indicator 11 comprises an α-MSH concentration in the normalised sample matrix 10. That is to say, the first analyte is α-MSH. The second FAS indicator 12 comprises a CLIP concentration in the normalised sample matrix 10. According to the present method the analytes from the sample matrix 10 are in each case extracted chromatographically and determined through subsequent mass spectrometry.

According to the embodiment shown in FIG. 3, the values of the first group of measured values 11n+ are compared with a first reference value 40 and the values of the second group of measured values 12+ are compared with a second reference value. The respective FAS indicator 11, 12 is now concluded on the basis of the respective comparison. Purely by way of example, the reference values 40, 50 shown in FIG. 3 lie above or substantially above the respective group of measured values 11n+, 12n+. Depending on the previous interpretation and definition, the reference values can, alternatively or additionally, also be lower, in particular also below the respective group of measured values, whereby the present result is nevertheless achieved. That is to say, in this case, a FAS indicator would lead to a meaningful result even if a group of measured values were above or essentially above a corresponding reference value. Thus, both an increased and a decreased FAS indicator can lead to the present FAS finding 30. Decisive, in particular, is the amount of the distance between the group of measured values and the reference value. This applies to all corresponding figures or associated embodiments.

According to FIG. 3, a positive first FAS indicator 11 can be assumed, since the expanded first group of measured values 11n+ is located in a range adjacent to the first reference value 40 and partly above this. Likewise, a positive second FAS indicator 12 can be assumed, since the expanded second group of measured values 12n+ is also located in a range adjacent to the second reference value 50 and partly above this. The FAS finding 30 is consequently also positive. That is to say, in this case a normal or substantially normal functioning or functionality of the anorexigenic signal path can be assumed. However, depending on the specification regarding the interpretation of the respective group of measured values 11n+, 12n+, the result represented in FIG. 3 could also be interpreted to the effect that the first FAS indicator 11 and the second FAS indicator 12 are in each case evaluated negatively, since an insufficient number of measured values 11n, 12n lie above the respective reference value 40, 50. As mentioned above, the overall consideration of the indicator spectrum according to predefined specifications is decisive.

In the fourth exemplary embodiment shown in FIG. 4, a quantitative mean first deviation value 60 of the first group of measured values 11n+ from the predefined first reference value 40 and a quantitative mean second deviation value 70 of the second group of measured values 12n+ from the predefined second reference value 50 are determined, and the FAS finding 30 is generated as a function of the first deviation value 60 and the second deviation value 70. With regard to the first group of measured values 11n+, it can be seen that although it lies relatively close to the first reference value 40, it is not close enough. Therefore, a correspondingly negative value is determined for the first FAS indicator 11. The second group of measured values 12n+ is relatively far from the second reference value 50, for which reason the second FAS indicator is also evaluated negatively. This also results in a negative overall result in the sense of a corresponding FAS finding 30.

In addition to the embodiments illustrated, the invention allows for further design principles. Thus, the blood sample can be provided as a liquid blood sample or as a dried blood sample. A whole blood sample, a plasma sample, a serum sample, a cerebrospinal fluid sample and/or a urine sample, in each case in liquid or dry form, can also be used as sample matrix 10. The first FAS indicator 11, the second FAS indicator 12 or the third FAS indicator 13 can comprise the concentration of at least one peptide hormone or in each case the concentration of different peptide hormones in the normalised sample matrix 10. The first FAS indicator 11, the second FAS indicator 12 and/or the third FAS indicator 13 can be multiplied by a weighting factor, wherein the FAS finding 30 is generated as a function of the weighted FAS indicators 11, 12, 13. In general, the first FAS indicator 11 can also be understood as second FAS indicator 12 or third FAS indicator 13, and vice versa. Furthermore, a plurality of first, second and/or third FAS indicators 11, 12, 13 can in each case be determined. The fasting and the exclusion of light can also last for a significantly shorter period.

### LIST OF REFERENCE SIGNS

1 patient

10 sample matrix

11 first FAS indicator

11n first measured value

11n+ first group of measured values

12 second FAS indicator

12n second measured value

12n+ second group of measured values

13 third FAS indicator

20 indicator spectrum

30 FAS finding

40 first reference value

50 second reference value

60 mean first deviation value

70 mean second deviation value

1. Method of providing a FAS finding (30) for the functionality of an anorexigenic signal path for a patient (1), comprising the steps:
placing the patient (1) in a normalised preparation state in preparation for a normalised sample collection,
providing a normalised sample matrix (10) collected from a patient (1) who was in the normalised preparation state, and
determining at least one FAS indicator (11, 12, 13) from the normalised sample matrix (10),
generating the FAS finding (30) based on the at least one determined FAS indicator (11, 12, 13).

2. Method according to claim 1,
characterised in that
in preparation for the sample collection, the patient (1) is subjected for a defined period of time to an exclusion of light, in particular an exclusion of sunlight.

3. Method according to claim 2,
characterised in that
the exclusion of light is carried out for at least 10 hours, in particular for at least 20 hours.

4. Method according to claim 1,
characterised in that
the patient (1) is placed in the normalised preparation state in preparation for a normalised blood sample collection, wherein the normalised sample matrix (10) is a plasma sample.

5. Method according to claim 1,
characterised in that
in preparation for the sample collection, the patient (1) is required to fast for a defined period of time.

6. Method according to claim 5,
characterised in that
the fasting is carried out for at least 6 hours, in particular within a time window of 12 hours to 36 hours.

7. Method according to claim 1,
characterised in that
the at least one FAS indicator (11, 12) is determined on the basis of at least one measured value (11n, 12n) for an analyte in the sample matrix.

8. Method according to claim 7,
characterised in that
the analyte is extracted from the sample matrix (10) chromatographically or by immunoprecipitation and determined by a subsequent mass spectrometry.

9. Method according to claim 7,
characterised in that
the analyte is MSH, in particular α-MSH.

10. Method according to claim 1,
characterised in that the at least one FAS indicator (11, 12) comprises a CLIP concentration in the normalised sample matrix (10).

11. Method according to claim 1,
characterised in that
the at least one FAS indicator (11, 12) comprises the concentration of at least one peptide hormone in the normalised sample matrix (10).

12. Method according to claim 1,
characterised in that
the at least one FAS indicator (11, 12) in each case comprises the concentration of different peptide hormones in the sample matrix (10).
